(19) United States
(12) Patent Application Publication
(10) Pub. No.: US 2019/0046085 A1
(43) Pub. Date: Feb. 14, 2019

(54) TREATMENT PROTOCOLS BASED ON PATIENT MOTION

(71) Applicant: Baylor Research Institute, Dallas, TX (US)

(72) Inventors: Yair M. Levy, Dallas, TX (US); Houston Holmes, Dallas, TX (US)

(21) Appl. No.: 15/574,110

(22) PCT Filed: May 11, 2016
(86) PCT No.: PCT/US16/31875

§ 371 Date: Nov. 14, 2017

Related U.S. Application Data
(60) Provisional application No. 62/162,433, filed on May 15, 2015.

(51) Int. Cl. A61B 5/11; A61B 5/00; G16H 50/20

(52) U.S. Cl. CPC A61B 5/1118; A61B 5/4836; G16H 50/20; A61B 5/7275

(57) ABSTRACT

A system for improving the treatment of a patient's disease. A patient is fitted with a wireless motion capture device. A computer system receives the captured motion data other patient metric information and compares same to stored data sets. The stored data sets may comprise other motion data and/or metric data from the patient being examined, or captured data from other patients. The stored data sets can also be tagged with, or otherwise associated with, one or more treatment protocols. A model can be formed with respect to the patient's captured motion and other metric information. A probable treatment of the diseases, effectiveness of the patient's treatment regimen, and progression of the disease itself can be ascertained from the comparison.

### CROSS-REFERENCE TO RELATED APPLICATION

This application claims the benefit of U.S. Provisional Patent Application No. 62/162,433, entitled, “TREATMENT PROTOCOL BASED ON PATIENT MOTION,” filed on May 15, 2015, which is expressly incorporated by reference herein in its entirety.

### TECHNICAL FIELD

The present disclosure relates to medical treatment technology. Specifically, the present disclosure relates to using captured patient motion data to improve treatment protocols.

### BACKGROUND

When treating a disease, a patient's “performance status” can be evaluated to measure the effectiveness of the treatment protocol assigned to that patient. A patient's performance status is commonly regarded as an aggregate profile of physical, physiological, and psychological effects of a disease. For example, the Eastern Cooperative Oncology Group (ECOG) performance status tool is one of the most widely used stratification tools in oncology clinical trials. ECOG performance status scores have been shown repeatedly to be relevant in the survival prognosis of cancer patients.

The ECOG performance status tool is simpler than its forerunner, i.e., the 11-point Karnofsky scale, which is meant to provide another measure of performance status. The Karnofsky scale requires significant effort to complete and only sometimes produces a reasonably valid and reliable metric.

Problematically, both the ECOG and Karnofsky scales rely on subjective assessments, based primarily on the patient and caregiver's opinion of degree of physical mobility. Correspondence of the two scales has been examined based on hit rate and weighted kappa coefficients of 1385 solid tumor patient assessments by 7 physicians within an oncology palliative care program. Agreement between scales is nonetheless far from ideal. This can be seen at Table 1, which illustrates the correspondence of a patient's performance status provided by the ECOG and Karnofsky Scales:

TABLE 1

| | | KPS Level |
|---|---|---|
| ECOG Scale | | (%^{a}) |
| Fully active, no restriction in pre-disease | 0 | 100 (39%) |
| performance | | |
| Restricted in physically strenuous activity but | 1 | 80-90 (65%) |
| ambulatory and able to carry out light work | | |
| Ambulatory; capable of all self-care but | 2 | 60-70 (90%) |
| unable to work; up more than 50% of waking | | |
| hours | | |
| Capable of only limited self-care; confined to | 3 | 40-50 (78%) |
| bed/chair >50% waking hours | | |
| Capable of only self-care; totally confined to | 4 | 10-30 (63%) |
| bed/chair | | |
| Dead | 5 | |

^{a}% of ECOG scores correctly predicted by KPS; according to study by Ma, et al [5].

Treatment protocols, e.g., protocols for treating cancer, are often toxic and frequently given with palliative rather than curative intent. As such, additional health-related quality of life measurements have also been advocated as additional metrics in cancer trials. Patient-reported outcome surveys in quality of life instruments are meant to provide information relating to physical pain, including side effects, as well as psychological well-being and the extent the patient perceives treatment as meeting or not meeting expectations.

Analysis of some clinical trial studies show that patient-reported outcome surveys can provide distinct prognostic information. Nevertheless, quality of life surveys and patient reported outcome surveys are subjective, and therefore, share the same limitations. For example, there is currently no standardized or objective patient-reported survey or an established scale for each applicable parameter or patient stratification. Therefore, the use of objective measurements could improve the accuracy of basic metrics such as the ECOG performance status scores or patient-reported outcome surveys used in developing a treatment protocol for a patient.

### DESCRIPTION OF THE FIGURES

For a more complete understanding of the concepts described herein, reference is now made to the following descriptions taken in conjunction with the accompanying drawings, in which:

FIG. 1 illustrates a treatment protocol system according to an embodiment;

FIG. 2 illustrates a treatment protocol method according to an embodiment;

FIG. 3 illustrates a treatment protocol method according to another embodiment; and

FIG. 4 illustrates a treatment protocol module according to an embodiment.

### DETAILED DESCRIPTION

In view of the foregoing, a system is provided for predicting, evaluating, and updating a patient's treatment protocol. Data indicative of the patient's activity can be used (1) as a surrogate indicator of pre-treatment physical well-being alongside standard of care clinical evaluations, and (2) to improve the accuracy of pre-treatment patient performance evaluations. A low cost, non-invasive wireless monitoring device is used to continuously measure and document the patient's pre- and/or post-treatment physical activity. The wireless device measures a patient's activity and transmits related data on a real-time, near real-time, or periodic basis, which is used as an input to objectively assess a patient's treatment. The patient's level of physical activity can be scored or stratified and used as a metric to determine an initial treatment protocol and/or modify a previously developed treatment protocol. For instance, the patient's physical activity level can be used as an objective metric applied as a pretreatment prognosticator for a cancer patient's response to chemotherapy (in terms of, e.g., toxicity, dose reductions, and delays). The patient's physical activity level can also be used as a metric to evaluate and/or predict patient bounce-back, hospital readmittance, discharge, and the like. Further, as discussed herein, the system can utilize a patient's physical activity level as a metric for developing a treatment protocol to treat different types of diseases. By way of example, a heart disease patient may be classified or scored according to level of physical activity, where the patient's score is correlated with the likely severity of heart disease.

A patient is fitted with a wireless monitoring device that comprises a number of sensors placed around a patient's body part, e.g., a wrist or ankle. The patient executes a number of physical activities, whether prescribed or not. While doing so, the wireless device transmits signals indicative of the patient's motion, which itself is directly related to the patient's activity level. A computer system receives the captured motion data and compares same to stored data sets. The stored data sets may comprise other motion data from the patient being treated, or captured data from other patients. The stored data sets can also be tagged with, or otherwise associated with, a diagnosis. Based upon the comparison, a model can be formed with respect to the patient's captured motion data. Meaningful information, such as the effectiveness of the patient's treatment regimen and progression of the patient's recovery can be ascertained from the comparison.

Embodiments provide real-time, near real-time, or periodic data indicative of a patient's physical activity level. This data can be correlated with other metrics and used to give healthcare providers information needed to predict an appropriate treatment for a patient and objectively measure the effectiveness of the treatment, while considering the patient's quality of life. Accordingly, described embodiments improve the treatment of various diseases. These improvements will reduce health care costs by allowing for more accurate and objective descriptions of effective treatment protocols and standardizing the treatments of diseases across different levels of healthcare providers.

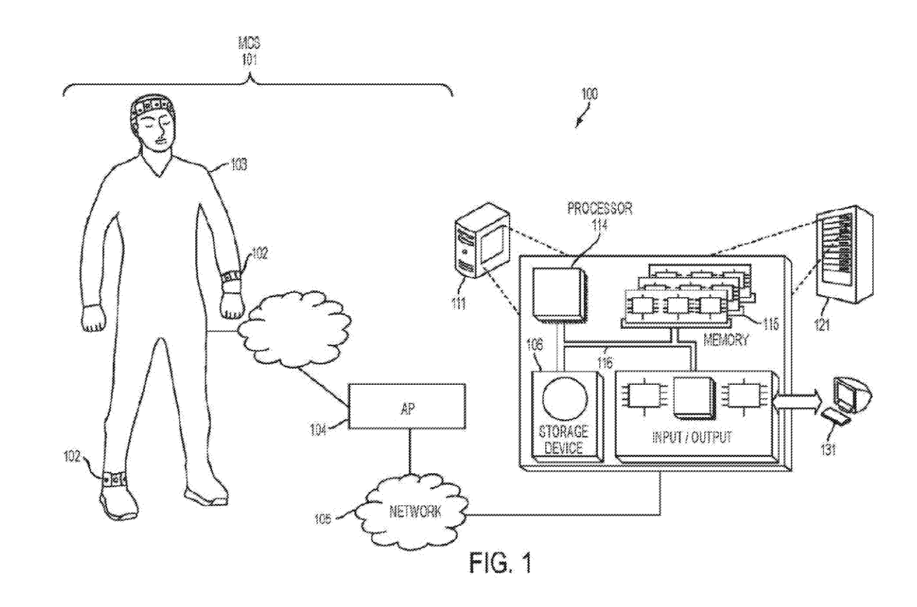

FIG. 1 illustrates an embodiment of disease treatment system 100. The illustrated embodiment comprises motion capture system 101, diagnostic computer 111, database 121, and graphical user interface 131. The components of system 100 can be implemented at a central facility to aggregate diagnostic data and offer services to users of system 100, or distributed across one or more networks to, e.g., improve capacity and data load balancing across system 100 and associated networks.

Motion capture system (MCS) 101 includes motion capture device 102 placed on patient 103. In some embodiments, MCS 101 may also include a base station or access point 104 paired with motion capture device 102. Different types of motion capture devices 102 can be placed around various locations of the patient's body to provide a user with specific data showing the patient's movements over a certain time period. Motion capture device 102 can be made in various sizes and have multiple adjustments to accommodate a sufficient fit for patients of various sizes and shapes. Motion capture device 102 can be customized to optimize its fit around varying body parts, including a patient's waist, ankle, wrist, and the like. Motion capture device 102 can be made of a comfortable and flexible material to facilitate unencumbered motion by patient 103 during analysis. Accordingly, in some embodiments, motion capture device 102 comprises a polymer-type material, or other flexible material, that may be worn around the wrist, ankle, or any appendage thereof.

Motion capture device 102 can be implemented as a band or bracelet that wraps around patient 103 at particular locations such as the wrist or ankle of patient 103. In some embodiments motion capture device 102 can be a single use, disposable type device. Such an embodiment is advantageous because it lowers the cost of capture device 102, and allows a patient to easily replace capture device 102 during different monitoring periods. However, it should be appreciated that, in some cases, capture device 102 should be configured so that is cannot easily be removed from the patient's body. The effectiveness of system 100 largely depends on the accuracy and precision of information captured by capture device 102. Therefore, should patient 103 remove capture device 102 for some length of time, doing so could have a detrimental impact on system 100's ability to develop an effective treatment protocol for patient 103. In cases where patient 103 is thought to likely remove captured device 102, it may be configured to make doing so difficult and/or be configured with an alert system that is activated upon detecting that it has been tampered with or otherwise modified from its original configuration.

Motion capture device 102 can continuously measure and document the patient's daily mobility. Motion capture device 102 monitors the number of steps the patient takes, distance traversed, and calories burned based on vigor of mobility and sleep quality, etc. Signals emitted by a built-in OLED (organic light-emitting diode) provide biofeedback to the user regarding extent of daily activity.

It should be appreciated that a variety of systems can be used to detect the motion of the patient 103. In one embodiment, motion capture device 102 automatically downloads acquired data when the user passes within a certain distance of a paired base station or access point 104. Data relating to the patient's motion over a certain time period is then fed into modeling software or the like executing on diagnostic computer 111. These measured parameters parallel and can add to other scoring criteria, e.g., the ECOG performance status scale, by providing objective data on the patient's robustness of mobility and sleep pattern.

Further, motion capture device 102 can be covered with or composed of a luminous material, or can be self-illuminating, such as motion capture device 102 that incorporates light emitting diodes (LEDs). For example, infra-red, self-illuminating motion capture device 102 can be seen when in dark environments, and, as a result, provides invariance to lighting conditions. Therefore, luminous or illuminated motion capture device 102 may facilitate performing motion capture and patient feedback when patient 103 is in either a light or dark environment.

In some embodiments, motion capture device 102 can be implemented with coded indicia that correspond to the identity of the patient. This coded identifying information is transmitted along with motion data. One advantage of using motion capture device 102 with coded indicia is that the indicia can be used to uniquely identify each patient 103 while complying with applicable privacy requirements. For example, a computer in communication with motion capture device 102 (e.g., diagnostic computer 111) can include an index that correlates observed coded indicia with particular motion capture device 102 worn by a particular patient 103. When the indicia are transmitted, the index can be accessed to locate the patient 103 associated with the coded indicia. In this way, treatment information can be retrieved for a given patient without requiring that personal information of the patient be revealed.

Motion capture device 102 and/or its paired access point 104 (if used in a given embodiment) can include memory to digitally record captured motion data as well as telemetry and transceiver software and hardware to transmit data structures comprising motion data and other information to diagnostic computer 111. This enables MCS 101 and other system components to wirelessly communicate with one another. As such, additional information including updates, control data, system performance and diagnostic data, and the like, can be communicated between system components. This is particularly advantageous for embodiments where system 100 is distributed, where, e.g., portions of diagnostic computer 111 reside on one or more mobile devices.

Motion capture device 102 may comprise inertial systems such as accelerometers, gyroscopes, and the like, as well as data inputs and data outputs. According to such an embodiment, motion capture device 102 transmits position data to other system components (e.g., access point 104 or camera 105), where the data is recorded and can be processed to meaningfully interpret the data. In such embodiments, motion capture device 102 and access point 104 each function as a wireless data transceiver, operating to receive data structures and process them accordingly.

Motion capture device 102 may comprise hardware and software enabling other properties associated with patient 103 to be sensed and communicated to other system components. Motion capture device 102 may also comprise a pressure sensor and/or a temperature sensor. Similarly, magnetic sensors can be used to transmit motion and/or position information. Each motion capture device 102 may further include a vibration transducer which can be activated by a local processor. The vibration transducer can be used to provide a sensory input at a certain physical position or orientations of sensors. For example, the vibration transducer can vibrate when patient 103 adopts an incorrect posture. If each motion capture device 102 is equipped with a vibration transducer, the body part or parts that are in the incorrect position can be indicated by means of the vibration transducer.

Motion capture device 102 may also comprise sensors that detect other metrics or qualities associated with patient 103. For example, motion capture device 102 may comprise sensors that detect physiological signals indicative of blood chemistry, blood sugar level, blood oxygen level, blood flow rate, heart rate, blood pressure, body temperature, and the like. Software executing in conjunction with the sensors may monitor these qualities or metrics over time and report them, or changes therein, when physical activity is reported by capture device 102. Further, when a quality or metric of interest is associated with a threshold or other significant level, capture device 102 may transmit an alert, both to other components of system 100 and/or patient 103, notifying each that the metric has exceeded the determined threshold. Such an application may be particularly important where, e.g., a heart disease patient is particularly interested in monitoring blood pressure and related functions.

Motion capture device 102, in some embodiments, can transmit the patient's physical activity level and other patient metrics aperiodically, and out of sync. That is, motion capture device 102 can transmit physical activity at periodic intervals but transmit other patient metrics only when, e.g., one or metrics exceeds a threshold. If, however, no threshold is exceeded, motion capture device 102 can synchronously transmit physical activity level and other patient metrics, and system 100 may develop a profile for each metric over time. Notably, the other patient metrics may be included as additional patient metrics that are used in conjunction with physical activity level to develop a treatment protocol for patient 103. In this regard, the combination of physical activity level and other patient metrics are leveraged to increase the effectiveness of system 100.

Also, each motion capture device 102 can have a unique ID. Accordingly, each motion capture device 102 can be connected to one or more access points 104 to essentially form a local area network. When located at or on motion capture device 102, each access point 104 can wirelessly transmit motion data, system performance data, and the like to communicate sensed data to a diagnostic computer 111. Motion capture device 102 and access points 104 comply with the applicable communication protocols and communicate with diagnostic computer 111 via network 105, which, preferably, is a local wireless network. Access points 104 preferably include a USB port or similar data interface when providing a wired connection to motion capture device 102. Likewise, motion capture device 102 can be powered centrally from access points 104 using the same interface that carries the data.

Each access point 104 can receive data signals from motion capture device 102 through a wired or wireless connection and transmit that data to a controller, which can be collocated with an access point 104, located at diagnostic computer 111, or positioned at a separate location. Access point 104 can also transmit diagnostic data, control data, and system data such as, e.g., battery life information, system performance, and the like, to the controller. In doing so, access point 104 may stream all data from motion capture device 102 to the controller in real-time or near real-time, or may transmit that data on a periodic basis, e.g., in response to a call for data from the controller. Further, multiple access points 104 can be provided, each having a user-configurable IP address. This allows multiple access points 104 to be controlled by a single controller. It also allows access points 104 to be controlled via the internet from a remote diagnostic computer 111.

Diagnostic computer 111 processes motion data captured by MCS 101. In doing so, hardware, software, and logic algorithms executing at diagnostic computer 111 can be utilized to gather and transform data associated with movements of patient 103 over a certain time period. Data structures comprising motion capture data are received at MCS interface 112.

The motion of patient 103 during a time period can be compared to several different sets of data and for different purposes. For example, patient 103's motion can be interpreted as an overall activity level, or broken down into discrete periods of activity within the time period. Each can be compared to stored benchmarks or stratified levels of activity modeled in software. This can be used to determine the relative activity of patient 103 and provide a score or other value.

Patient 103's motion can also be compared to patient 103's motion captured during other time periods. This can be used to evaluate patient 103's progress between capture sessions and/or the effectiveness of the current treatment regimen. Patient 103's motion can also be compared to that of another patient. This can be used to compare patient 103's motion to that of a healthy person to provide a predictive treatment protocol.

In any event, the data ultimately compared to patient 103's captured motion can be stored as sets of data in database 121. For example, if the stored data sets used for comparison related to motion data captured from other patients, the data sets can be tagged with, or otherwise associated with, a diagnosis or other metric relating to the patient. If, for example, a patient is diagnosed with a certain disease and is determined to undergo a certain level of activity over a given time period, that information can be compiled to form a test profile. That test profile can be compared to another data set, or profile, compiled from the examination of another patient. If the diagnosis and level of activity of patient 103's test profile match or substantially overlap with that from a stored test profile relating to another patient, then the treatment associated with the stored test profile may then be applied to or associated with patient 103's test profile. Once this association is made, the healthcare provider can share the treatment with patient 103 and develop an appropriate treatment protocol.

Further, real-time diagnostic information can be provided and displayed on graphical user interface (GUI) 131 or a similar display in a standalone application or via a web based system using a web server. The display can incorporate without limitation 3D medical anatomical displays, biomechanical data interpretation, and interactive imaging that is needed for the diagnosis and/or treatment of patient 103. Using GUI 131, diagnostic computer 111 may provide a healthcare provider with a diagnostic solution that includes visual and verbal guidance instructions directing the steps needed for treating the patient. In some embodiments, the logic algorithms can visually and verbally guide the healthcare provider in a step by step process to treat patient 103's dysfunctions.

Diagnostic computer 111 can be used to implement processing functionality for various aspects of developing a treatment. Diagnostic computer 111 may comprise, e.g., a user device such as a desktop, mobile device, a mainframe, server, or any other type of special or general purpose computing device as can be desirable or appropriate for the given application or environment. Diagnostic computer 111 can include one or more processors, such as a processor 114. Processor 114 can be implemented using a general or special purpose processing engine such as, for example, a microprocessor, microcontroller or other control logic. In this example, processor 114 is connected to a bus 116 or other communication medium.

Diagnostic computer 111 can also include a main memory 115, such as random access memory (RAM) or other dynamic memory, for storing information and instructions to be executed by processor 114. Main memory 115 also can be used for storing temporary variables or other intermediate information during execution of instructions to be executed by processor 114. Diagnostic computer 111 may likewise include a read only memory (ROM) or other static storage device (not shown) coupled to bus 116 for storing static information and instructions for processor 114.

Diagnostic computer 111 may also include media drive 117 and removable storage media 118. Media drive 117 can include a drive or other mechanism to support fixed or removable storage media, such as a hard disk drive, floppy disk drive, magnetic tape drive, optical disk drive, CD or DVD drive (R or RW), or other removable or fixed media drive. Storage media 118 can include, for example, a hard disk, floppy disk, magnetic tape, optical disk, CD or DVD, or other fixed or removable medium that is read by and written to by media drive 117. As these examples illustrate, the storage media 118 can include a computer-readable storage medium having stored thereon particular computer software or data.

Diagnostic computer 111 can include other similar instrumentalities for allowing computer programs or other instructions or data to be loaded into diagnostic computer 111. Such instrumentalities can include, for example, a removable storage unit, and an interface, such as a program cartridge and cartridge interface, a removable memory (for example, a flash memory or other removable memory module) and memory slot, and other removable storage units that allow software and data to be transferred from the storage media 118 to diagnostic computer 111.

Diagnostic computer 111 can also include communications interfaces 112 and 113. Communications interfaces 112 and 113 can be used to allow software and data to be transferred between diagnostic computer 111 and external devices. Examples of communications interfaces 112 and 113 can include a modem, a network interface (such as an Ethernet or other NIC card), a communications port (such as for example, a USB port), a PCMCIA slot and card, etc. Software and data transferred via communications interfaces 112 and 113 are in the form of signals which can be electronic, electromagnetic, optical, or other signals capable of being received by communications interfaces 112 and 113. These signals are provided to communications interfaces 112 and 113 via a channel 119. Channel 119 may carry signals and can be implemented using a wireless medium, wire or cable, fiber optics, or other communications medium. Some examples of a channel include a phone line, a cellular phone link, an RF link, a network interface, a local or wide area network, and other communications channels. Essentially any physical layer connectivity and communications protocol can be employed. In some embodiments, signals are conducted wirelessly, and may employ any suitable carrier frequency, modulation scheme and protocol, such as BLUETOOTH®, ZigBee®, CDMA, GSM and the like. Data can be stored and accessed in any manner known to the art, including local storage, remote servers and cloud computing.

According to one embodiment, the components of diagnostic computer 111 are distributed. In such an embodiment, GUI 131 can be implemented on a user's mobile device or the like and functionality can be provided by an application installed on the user's mobile device. Information can be shared across system 100 as one user's mobile device communicates information with other components, including database 121 and mobile devices associated with other users. Such an embodiment is advantageous because it allows one healthcare provider to access information relating to patient 103 and share that information with patient 103 and/or other healthcare providers, while requiring only mobile devices. In such embodiments, information is encrypted to ensure privacy is protected and system 100 complies with applicable regulatory provisions. Naturally, each healthcare provider and patient 103 can be required to submit validation credentials to access the information provided by system 100.

The terms “computer program product” and “computer-readable storage medium” can be used generally to refer to media. These and other forms of computer-readable storage media can be involved in providing one or more sequences of one or more instructions to processor 114 for execution. Such instructions, generally referred to as “computer program code” (which can be grouped in the form of computer programs or other groupings), when executed, enable the diagnostic computer 111 to perform features or functions of embodiments of the current technology.

In an embodiment where the elements are implemented using software, the software can be stored in a computer-readable storage medium and loaded into diagnostic computer 111 using, e.g., removable storage drive 118 or communications interfaces 112 and 113. The control logic (in this example, software instructions or computer program code), when executed by the processor 114, causes the processor 114 to perform the functions of the technology as described herein.

It should be appreciated that, in some embodiments, diagnostic computer 111 (or at least some components thereof) may reside and execute upon a mobile device, such as a smart phone and the like. In that case, a user may interface with diagnostic computer 111 via the smart phone interface, and use functionality provided by diagnostic computer by, e.g., calling an application that executes on the user's smartphone. In that case, diagnostic computer 111 leverages hardware and memory residing on the smartphone to execute the processes described herein.

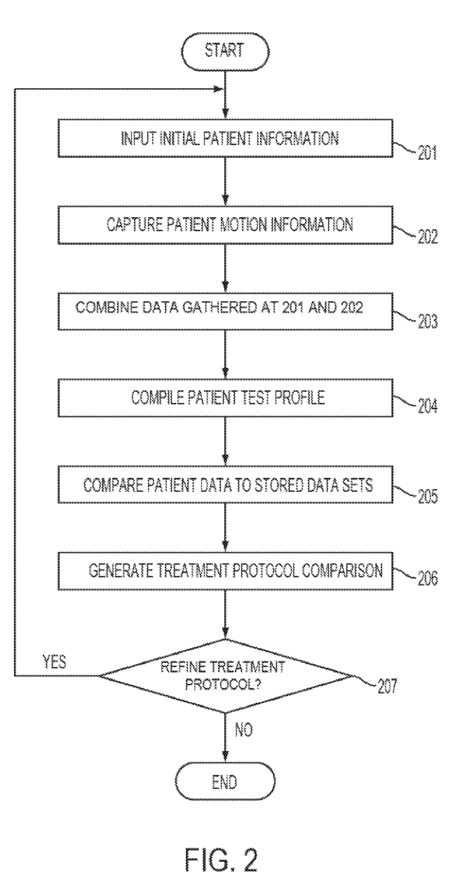

FIG. 2 is a flow chart illustrating steps in a method for developing a treatment based on the motion of a patient. Patient treatment method 200 begins with an assessment of the patient's information. At step 201, initial data relating to the patient is input to a diagnostic computer, such as, e.g., diagnostic computer 111 illustrated at FIG. 1. The initial data may comprise a patient diagnosis, height, weight, sex, age, body mass index (BMI), body fat percentage, appendage circumference, and the like. Also, additional information can be entered including general medical history, medical history associated with one or more problematic joints, current treatment regimens, and imaging information (e.g., MRI information). This information can be considered as part of the treatment protocol.

At step 202, the patient's motion is monitored over a period of time. That is, the patient executes biomechanical movements over a period of time, which provides a meaningful picture of the patient's level of activity. A wireless device worn by the patient monitors the patient's motion and can transmit the data periodically or stream the data to one or more access points 104 (if in a predetermined range) or directly to another system component over one or more networks.

At step 203, captured motion data is combined with the initial data provide at step 201 to form a patient profile. Further, the captured motion and initial data may be augmented with additional information. This information may be input by a user or a patient and may serve to add further context to the patient's quality of life or condition. For example, notations relating to pain, sleep quality, heart rate, respiration rate, blood glucose level, and the like may be considered as part of the treatment protocol. Overlaying the information with the captured motion information provides further meaning to the patient's likely treatment. At step 204, a patient test profile is optionally created from the initial data received at step 201, the motion data captured as step 202, and any additional data received at step 203. The profile can be compiled by hardware and software residing at, e.g., diagnostic computer 111 illustrated at FIG. 1.

At step 205, a comparison is performed between the patient's captured motion data stored data sets. This can be done by comparing the data in its raw form, or by comparing the data as compiled in a patient test profile, as described with respect to preceding optional step 204. Further, the inclusion of additional data captured at step 203 is optional. So, in the event additional data is captured from the patient, but not included in the stored data sets, that information can be omitted from the comparison.

As discussed, the patient's motion can be compared to several different sets of data and for different purposes. The patient's motion can be compared to patterns modeled in software to depict idealized motion or activity levels. A score or activity level category can be assigned to the patient. This can be used to determine the characterization of the patient's treatment. The patient's motion or activity level can also be compared to his or her motion or activity level stored from other motion capture sessions. This can be used to evaluate the patient's progress between capture sessions and/or the effectiveness of the current treatment regimen. The patient's motion can also be compared to that of another patient. This can be used to compare the patient's motion to that of a healthy person to provide a treatment. When the stored data sets used for comparison relate to motion data captured from other patients, the data sets can be tagged with, or otherwise associated with, a diagnosis or other metric.

At step 206, based upon the comparison, a treatment protocol (or probable treatment protocol) is provided. By way of example, if the patient displays particular levels activity after being diagnosed with a certain condition, that information can be compiled to form a test profile. That test profile can be compared to another data set, or profile, compiled from the examination of another patient. If the diagnosis and activity level in the patient's test profile match or substantially overlap with that from a stored test profile relating to another patient, then the diagnosis associated with the stored test profile may then be attributed to the patient's test profile. Once this association is made, the healthcare provider can share the diagnosis with the patient and develop an appropriate treatment protocol.

At step 207, a refinement of the initial treatment can be performed. In that case, subsequent motion data can be acquired, and the logic software can again analyze the patient's activity level. Accordingly, another dynamic motion capture can be performed and the modeling software can consider the changes therein to modify the recommended treatment.

Consistent with the foregoing, it is envisaged that system 100 can be utilized as a predicator for patient bounce-back, i.e., hospital readmittance. Therefore, system 100 may be used to provide a reliable indication if a patient is ready for discharge thereby reducing bounce-back and reducing hospital fees. As mentioned, system 100 can be applied different fields. In addition to the fields of oncology, system 100 may be utilized for, e.g., cardiology patients and/or general surgical fields as a means of predicating if the patient is a viable candidate for the proposed treatment plan and/or if the patient is ready for discharge after proposed treatment has been implemented.

### Example

Consider the case where a patient is diagnosed with cancer. The patient will undergo a preliminary evaluation in which vital information such as BMI, medical information such as medical history, and imaging information such as CT images will be input to the diagnostic computer for consideration in the treatment protocol. Based on information collected from the patient, an initial performance status is assigned to the patient.

The patient will then be fitted with a wireless motion monitoring device around the waist, ankle, or wrist. Typically, a patient will not be made aware of the value of the motion data to minimize the likelihood of the patient modifying activity level and skewing the results. A healthcare provider will note the time and date at which the patient begins wearing the wireless monitoring device.

Going forward, the patient's motion can be captured by the motion capture device, which will concurrently transmit data structures indicative of motion or activity level during the tests. The data structures can be transmitted via a wired or wireless interface and communication protocol.

Once the data is received, the diagnostic computer executes steps to compute a treatment protocol. The algorithm will process the recorded data to provide a possible treatment. The underlying process overlays the patient's level of activity received from the patient with other considerations such as diagnoses and information provided by the patient. As mentioned, the captured motion data can be compiled to form a patient profile. Data fields within the patient profile can be compared to other sets of stored data to associate or tag the patient's test profile with a treatment.

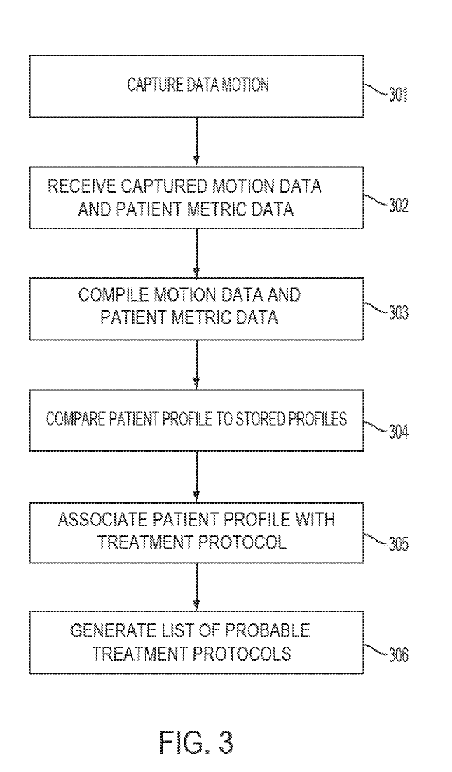

FIG. 3 illustrates functional blocks executed to predict, evaluate, and refine a treatment protocol according to the concepts described herein. Specifically, FIG. 3 illustrates functional blocks executed by, e.g., system components illustrated at FIG. 1, such as diagnostic computer 111. At step 301, a wireless motion capture device worn by a patient captures motion or other data indicative of activity level for one or selected periods of time. At step 302, a diagnostic computer in communication with the wireless motion capture device receives the captured motion data and patient metric data such as diagnosis, and perhaps additional contextual information provided by the patient.

At step 303, the diagnostic computer compiles the captured motion data and the patient metric data to generate a test patient profile. Generating the test patient profile may also comprise correlating the values of different received data. Generating the test patient profile may further comprise correlating additional information, e.g., the body mass index (BMI) of the test patient, with the captured motion data.

At step 304, the diagnostic computer compares the patient profile to one or more stored profiles. The one or more stored profiles comprises captured motion data as well as patient metric data. Each stored profile is associated with one or more probable treatments. Each probably treatment may further be associated with a predicted likelihood of success. At step 305, the test patient profile is associated with a treatment protocol, which is associated with the one or more stored profiles that matches the patient profile.

At step 306, the diagnostic computer generates a list of probable treatment protocols. The protocols may be ranked in order of predicted likelihood of success.

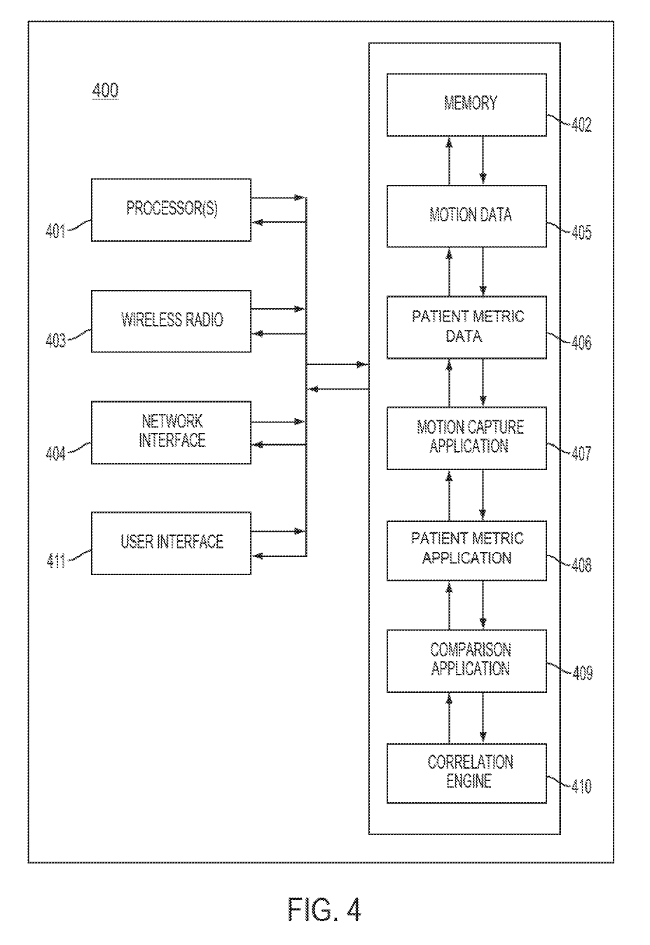

FIG. 4 illustrates, in more detail, components of an apparatus that enables development of a treatment protocol according to the concepts described herein. Referring to FIG. 4, treatment module 400 may correspond to diagnostic computer 111 illustrated in FIG. 1. Components of module 400 may comprise hardware, software, firmware, program code, or other logic (for example, ASIC, FPGA, etc.), as can be operable to provide the functions described herein. Module 400 comprises components that, when executing operations described herein, effectuate mechanisms for predicting, evaluating, and updating treatment protocols. Each of these components can be separate from, or integral with, module 400.

The functionality and operations of module 400 are controlled and executed through processor(s) 401 and specialized software executing thereon. Processor(s) 401 can include one or more core processors, central processing units (CPUs), graphical processing units (GPUs), math co-processors, and the like. Processor(s) 401 execute program logic, whether implemented through software stored in a memory 402 or in firmware in which logic is integrated directly into integrated circuit components. Module 400 may communicate wirelessly with other system components. Processor(s) 401 execute program logic, whether implemented through software stored in a memory 402 or in firmware in which logic is integrated directly into integrated circuit components. Module 400 may communicate wirelessly with other system components, such as multiple nodes comprising MCS 101, patient 103, and access point 104, through various radios, such as wireless radio 403, such as one or more of wireless wide area network (WWAN) radios and wireless local area network (WLAN) radios. If a WWAN radio is included as one of the radios in wireless radio 403, communication would generally be allowed over a long range wireless communication network such as 3G, 4G, LTE, and the like. Various WLAN radios, such as WIFI™ radios, BLUETOOTH® radios, and the like, would allow communication over a shorter range. Module 400 may also provide communication and network access through a wired connection with network interface 404. The wired connection may connect to the public-switched telephone network (PSTN), or other communication network, in order to connect to the Internet or other accessible communication network.

Module 400 can include memory 402 that stores signals that are used to transmit and receive data and control data, including patient diagnostic data. Memory 402 can be provided as a separate component and affixed to or formed integrally with module 400. The exact location of memory 402 is not limited and memory 402 can be provided with any form of communication means to communicate with MCS 101 and module 400. Memory 402 can include any type of data storage means such as computer readable memory or the like. Preferably, memory 402 stores captured motion data 405 and patient metric data 406.

Under control of processor(s) 401, program logic stored on memory 402, including motion capture application 407, patient metric application 408, comparison application 409, correlation engine 410, and other applications are executed to provide the functionality of module 400, including communications, storage, computation, filtering, analysis, and correlation of motion capture data and patient metric data, and transmitting data to system components to development of a treatment protocol. Various operating applications can be displayed visually to the user via user interface 411.

Motion capture application 407 extracts data structures indicative of a patient motion or level of activity over a given period of time, where the data is received from a wireless motion capture device or paired access point. Patient metric application 408 receives data structures indicative or one or more metrics associated with the patient.

Comparison application 409 compiles the captured motion data and the patient metrics to generate a patient profile. Afterward, comparison application 409 compares the test patient profile to one or more stored profiles, the one or more stored profiles comprising captured motion and metric information of another test patient. Each stored profile is associated with one or more treatment protocols that are thought to be successful given the activity level and metric information (e.g., patient diagnosis).

Based on the comparing, correlation engine 410 associates the test patient profile with a treatment protocol, which itself is associated with the one or more stored profiles that matches the patient profile.

The specific metrics and/or other variables considered by system 100 in developing a treatment protocol may be based on a particular clinical score that is generated. For example, with respect to developing a treatment protocol for a patient with cancer, the patient may be scored or categorized according to monitored physical activity level. The score or category assigned to the patient is then used as a factor in developing the treatment protocol. For example, the following chart illustrates scores that are assigned to a patient and activities and/or symptoms associated with the scores that are considered in developing a treatment protocol. By way of example, based on N=1 (one patient), a score of 0 corresponds to more than 2,000 steps/day, a score of 1 corresponds to 1,000-2,000 steps/day, a score of 2 corresponds to 500-1,000 steps/day, a score of 3 corresponds to less than 500 steps/day, and a score of 4, corresponds to less than 50 steps/day where, e.g., the patient spends the large majority of time in resting in bed or in a chair.

| Score | Expected Activity Level and Symptoms |
|---|---|
| 0 | Fully active, able to carry on all pre-disease performance without restriction |
| 1 | Restricted in physically strenuous activity but ambulatory and able to carry out |
| | work of a light or sedentary nature, e.g., light house work, office work |
| 2 | Ambulatory and capable of all self-care but unable to carry out any work |
| | activities. Up and about more than 50% of waking hours |
| 3 | Capable of only limited self-care, confined to bed or chair more than 50% of |
| | waking hours |
| 4 | Completely disabled. Cannot carry on any self-care. Totally confined to bed or |
| | chair |
| 5 | Dead |

Further, the recorded steps/activity can also be compared to self-reported measures of activity as well. Other data to be collected will include flights of stairs climbed, time at rest, and sleep, and the like.

1. A method for assigning a treatment protocol based on patient motion, the method comprising:
receiving, at one or more processors, data indicative of a patient's motion, where patient motion is perceived by a wireless device worn by the patient;
combining, using the one or more processors, the data indicative of the patient's motion with one or more metrics of the patient to generate a patient profile;
comparing, using the one or more processors, the generated patient profile to one or more stored patient profiles, the one or more stored patient profiles associated with a treatment protocol; and
based on the comparing, associating the generated patient profile with a treatment protocol associated with the one or more stored patient profiles that matches the generated patient profile.

2. The method of claim 1 further comprising:
assigning, to the patient, the treatment protocol associated with the generated patient profile.

3. The method of claim 1 further comprising:
receiving, at the one or more processors, second data indicative of the patient's motion;
combining, using the one or more processors, the second data indicative of the patient's motion with the one or more metrics of the patient to generate an updated patient profile;
comparing, using the one or more processors, the updated patient profile to the one or more stored patient profiles; and
based on the comparing, associating the updated patient profile with a treatment protocol associated with the one or more stored patient profiles that matches the updated patient profile.

4. The method of claim 2 further comprising:
assigning, to the patient, the treatment protocol associated with the updated patient profile.

5. The method of claim 1 where the one or more stored profiles comprise:
data indicative of another patient's motion and the one or more metrics of another patient.

6. (canceled)

7. The method of claim 1 where the one or more metrics of the patient comprises a patient diagnosis.

8. An apparatus for computing a treatment protocol, the system comprising:
a non-transitory memory; and
one or more processors in communication with the memory, the one or more processors configured to:
receive, at one or more processors, data indicative of a patient's motion, where patient motion is perceived by a wireless device worn by the patient;
combine, using the one or more processors, the data indicative of the patient's motion with one or more metrics of the patient to generate a patient profile;
compare, using the one or more processors, the generated patient profile to one or more stored patient profiles, the one or more stored patient profiles associated with a treatment protocol; and
based on the comparing, associating the generated patient profile with a treatment protocol associated with the one or more stored patient profiles that matches the generated patient profile.

9. The apparatus of claim 8 where the one or more processors are further configured to:
assign, to the patient, the treatment protocol associated with the generated patient profile.

10. The apparatus of claim 8 where the one or more processors are further configured to:
receive, at the one or more processors, second data indicative of the patient's motion;
combine, using the one or more processors, the second data indicative of the patient's motion with the one or more metrics of the patient to generate an updated patient profile;
compare, using the one or more processors, the updated patient profile to the one or more stored patient profiles; and
based on the comparing, associating the updated patient profile with a treatment protocol associated with the one or more stored patient profiles that matches the updated patient profile.

11. The apparatus of claim 9 where the one or more processors are further configured to:
assign, to the patient, the treatment protocol associated with the updated patient profile.

12. The apparatus of claim 8 where the one or more stored profiles comprise:
data indicative of another patient's motion and the one or more metrics of another patient.

13. The apparatus of claim 8 where the one or more processors are further configured to:
store, in non-transitory memory, the data indicative of patient motion.

14. (canceled)

15. A method for assigning a treatment protocol based on patient motion, the method comprising:
receiving, at one or more processors, data indicative of a patient's motion, where patient motion is perceived by a wireless device worn by the patient;
correlating, using the one or more processors, the data indicative of patient motion to a patient activity level;
combining the correlated patient activity level with one or more metrics of the patient to generate a patient profile; and
assigning, to the patient, a treatment protocol that corresponds to the generated patient profile.

16. The method of claim 15 further comprising:
receiving, at the one or more processors, second data indicative of the patient's motion;
correlating, using the one or more processors, the second data indicative of patient motion to a second patient activity level;
combining, using the one or more processors, the second patient activity level with the one or more metrics of the patient to generate an updated patient profile; and
assigning, to the patient, an updated treatment protocol based on the updated patient profile.

17. The method of claim 15 where correlating the data indicative of patient motion to a patient activity level comprises:
comparing the data indicative of patient motion to one or more thresholds, each one or more thresholds demarcating levels of patient activity.

18. The method of claim 15 further comprising:
storing, in non-transitory memory, the data indicative of patient motion.

19. (canceled)

20. An apparatus for computing a treatment protocol, the system comprising:
a non-transitory memory; and
one or more processors in communication with the memory, the one or more processors configured to:
receive, at one or more processors, data indicative of a patient's motion, where patient motion is perceived by a wireless device worn by the patient;
correlate, using the one or more processors, the data indicative of patient motion to a patient activity level;
combine the correlated patient activity level with one or more metrics of the patient to generate a patient profile; and
assigning, to the patient, a treatment protocol that corresponds to the generated patient profile.

21. The apparatus of claim 20 where the one or more processors are further configured to:
receive, at the one or more processors, second data indicative of the patient's motion;
correlate, using the one or more processors, the second data indicative of patient motion to a second patient activity level;
combine, using the one or more processors, the second patient activity level with the one or more metrics of the patient to generate an updated patient profile; and
assign, to the patient, an updated treatment protocol based on the updated patient profile.

22. The apparatus of claim 20 where the one or more processors are further configured to:
compare the data indicative of patient motion to one or more thresholds, each one or more thresholds demarcating levels of patient activity.

23. (canceled)

24. The apparatus of claim 20
where the one or more metrics of the patient comprises a patient diagnosis.
